(19) United States
(12) Patent Application Publication
(10) Pub. No.: US 2026/0012162 A1
(43) Pub. Date: Jan. 8, 2026

(54) OPTIMISATION OF CONTROL OF REACTIVE CIRCUIT FOR GENERATING ELECTROMAGNETIC PULSES

(71) Applicant: Oxrord University Innovation Limited, Oxford (GB)

(72) Inventors: Daniel ROGERS, Oxford (GB); Kawsar ALI, Oxford (GB)

(21) Appl. No.: 18/993,339

(22) PCT Filed: Jul. 5, 2023
(86) PCT No.: PCT/GB2023/051760

§ 371 Date: Jan. 10, 2025

(30) Foreign Application Priority Data
Jul. 11, 2022 (GB) 2210149.7

(51) Int. Cl. H03K 3/57; A61N 2/00; A61N 2/02; H02M 1/00; H02M 7/483

(52) U.S. Cl. CPC H03K 3/57; A61N 2/006; A61N 2/02; H02M 1/0012; H02M 7/4835

(57) ABSTRACT

Offline prior to operation, an optimised control sequence is derived for a pulse system comprising a reactive circuit for generating electromagnetic pulses, such as a transcranial magnetic stimulation circuit, and cascaded switching modules for switchably connecting capacitive discharge elements to provide a multi-level output voltage for the reactive circuit. A discrete-time model predicts an electrical state of the pulse system. A target profile for an electromagnetic pulse is received. For respective time-steps of the control sequence taken in succession, an optimal path of switching states over a window of time-steps starting with the respective time-step is derived, optimising a cost function having a deviation cost contribution representing deviation of an electrical state of the pulse system predicted by the discrete-time model from the target profile over the window. The switching state of the determined path at the respective time-step is selected for the control sequence.

The present invention relates to control of a pulse system comprising a reactive circuit including a pulse coil for generating electromagnetic pulses.

A pulse system for generating electromagnetic pulses using a pulse coil may comprise a switching circuit containing an arrangement of switches for switchably connecting capacitive discharge elements to provide an input to a reactive circuit that includes the pulse coil. Such a pulse system may be operated under the control of a control sequence that is a sequence of switching states of the switches over time-steps. In a typical arrangement, the switching circuit comprises a cascade of switching modules in which the switches are arranged to connect a respective capacitive discharge element across module outputs that are cascaded to provide a multi-level output voltage.

When controlling such a pulse system, typically the aim is to meet a target profile for the electromagnetic pulse, for example a target profile of current through the pulse coil. In practice this is difficult to achieve because of the complexity of the response of the reactive circuit to the continually varying output voltage derived from the capacitive discharge elements in the switching circuit as energy is transferred. The control sequence has many degrees of freedom in the choice and timing of switching of individual switches and it is not straightforward to predict what control system will optimise the response of the reactive circuit, which may for example include a complex filter circuit that filters the input to the reactive circuit before application to the pulse coil.

In addition, in some cases, this objective may be subject to meeting desired operational characteristics of the switching circuit, for example limiting the rate of switching of the switches, and maintaining a balance of the voltages of the capacitive discharge elements, among others.

According to a first aspect of the invention there is provided a method of deriving an optimised control sequence for a pulse system comprising a reactive circuit including a pulse coil for generating electromagnetic pulses, and a switching circuit comprising plural switches arranged to switchably connect at least one capacitive discharge element to provide an output voltage as an input to the reactive circuit, and the control sequence is a sequence of switching states of the switches over time-steps, wherein the method is performed offline prior to operating the pulse system under the control of the derived, optimised control sequence, and uses a discrete-time model of the pulse system configured to predict an electrical state of the pulse system, the method comprising: receiving a target profile for an electromagnetic pulse delivered by the pulse coil; and for respective time-steps of the control sequence taken in succession, determining an optimal path of switching states over a window of time-steps starting with the respective time-step, which optimal path of switching states optimises a cost function having a deviation cost contribution representing deviation of an electrical state of the pulse system predicted by the discrete-time model from the target profile over the window of time-steps, and selecting the switching state of the determined path at the respective time-step as the switching state of the control sequence at the respective time-step.

The advantage of this method is that it provides a systematic way to achieve electromagnetic pulses meeting the target profile to a high degree of accuracy. In particular, as the determined optimal path of switching states that optimises the cost function having a deviation cost contribution representing deviation of an electrical state of the pulse system predicted by the discrete-time model from the target profile, the optimised control sequence that is derived tends to minimise that deviation, allowing the target profile to be followed. The method can be readily adapted to any new pulse coil and/or filter by changing parameters in the discrete-time model without complex re-tuning.

This method of deriving an optimised control sequence may be considered as a modified type of model predictive control (MPC) algorithm, distinguished in one way by its offline nature. As control sequence is derived for any given target profile in advance of operating the pulse system under the control of the derived, optimised control sequence, the method eliminates the computational challenge of a notional alternative of performing control dynamically in real-time, which in practice and within the limits of available computational resource is difficult to perform, with the result that the derived control sequence does not allow the target profile to be accurately followed.

Preferably, the cost function may have further cost contributions that represent penalties for undesirable operational characteristics of the switching circuit. In this manner, the optimisation of the cost function also causes the optimised control sequence to optimise the operational characteristics of the switching circuit. Some specific examples are as follows.

Preferably, the at least one further cost contribution may include a voltage cost contribution that is dependent on the voltage or voltages of the at least one capacitive discharge element.

This has the advantage of allowing the method to consider the at least one capacitive discharge element of the switching circuit when calculating the cost.

Preferably, the voltage cost contribution represents a penalty for variation between the voltages of plural capacitive discharge element over the window.

This has the advantage of preventing variation in the capacitive discharge elements to minimise any deviation, ensuring that one or more capacitive discharge elements do not have a significantly higher voltage than the others.

Preferably, the voltage cost contribution takes a prohibitive value when the voltage of a capacitive discharge element exceeds an overcharge threshold.

This has the advantage of preventing damage to the capacitive discharge element due to high voltages, as the cost function will ensure that the method will never consider a path that causes the voltage to exceed an overcharge threshold, due to the cost being higher than any alternative path that does not exceed it.

Preferably, the at least one further cost contribution may include at least one switching state cost contribution that is dependent on the switching states of the switches.

This provides the advantage of allowing the method to consider the states of the switching circuit when calculating the cost.

Preferably, the at least one switching state cost contribution includes at least one switching action cost contribution representing penalties for undesirable switching actions.

This provides the advantage of allowing the method to avoid performing undesirable switching actions as alternative paths that do not perform these actions will have a lower cost and so be preferred over paths that do.

Preferably, the at least one switching action cost contribution comprises one of more of: a switching action cost contribution representing a penalty for switching states connecting the at least one capacitive discharge element to provide the output voltage; a switching action cost contribution representing a penalty for simultaneous change of the switching state of plural switches; and/or a switching action cost contribution representing a penalty for changes in the output voltage at each time-step.

This various switching action costs provide the advantage of preventing unnecessary changes in the state of the pulse system, such as performing unnecessary switches and ensuring that as few switches as needed change state. The cost will cause the method to prefer paths that make fewer changes in state, when compared with paths that achieve the same effect but with more changes in the system state. This leads to a more reliable system as the components are changing state less and so less stress is being placed on the individual components.

Preferably, the at least one switching state cost contribution includes an equalisation cost contribution representing a penalty for unequal switching counts of different switches.

The advantage of this is that it allows the method to account for the number of times switches in the switching circuit been switched by increasing the cost when some switches are switched more than others. This prevents the switches of the switching circuit from becoming unequally stressed due to one performing a significantly higher amount of switching. Unequal switching could damage the components and so this leads to a more reliable system as the load is spread more evenly.

The determination of an optimal path of switching states may comprise performing a search of possible paths of switching states over the window by selecting paths successively and: for an initially selected path, accumulating a total value of the cost function over the entirety of the path; for subsequently selected paths, accumulating a value of the cost function over successive time-steps of the path, wherein at each time-step before the final time-step, the accumulated value of the cost function is compared to a minimum total value of the cost function accumulated over the entirety of a path previously in the search, and if the accumulated value has reached the minimum value then the accumulation is terminated, and otherwise the accumulation is continued, and on completion of the search, determining that the path having a minimum total value of the cost function accumulated over the entirety of the path is the optimal path.

This provides the advantage of allowing the algorithm to evaluate the current path in comparison to the best known path discovered so far, reducing time wasted searching sub-optimal paths that will not provide a more optimal solution. If the current path has a greater than or equivalent accumulated value to the best known path, there is no advantage to searching the current path any further, as the accumulated cost will only ever increase further into the path. This allows for the time to determine the optimal path to be decreased, by discarding a path once it is clear that it will not lead to the optimal path, and immediately beginning to search the next possible path. Storing only the optimal path found so far, as opposed to all paths explored, also reduces the amount of memory the algorithm requires.

Preferably, for respective time-steps of the control sequence subsequent to the first, the step of selecting paths successively comprises initially selecting paths that commence with the overlapping part of the optimal path that was determined for a previous time-step of the control sequence.

This provides the advantage of allowing the method to ensure that the initially selected path is likely to be a viable solution, reducing the number of branch evaluations and the time spent finding the optimal path. As the overlapping part of the optimal path was found to have the minimum cost value in the previous time-step, any paths that include it in this time-step are also likely to have a low cost value. This means that the initially selected path for this time-step is likely to have a low cost value, and so a large number of possible paths will be discarded early in the search process. These paths will also require little effort to explore, allowing an approximate viable solution to be found quickly. This leads to less of the search space being explored and so a reduction in the time spent finding the optimal path.

Preferably, at an early stage of the search, the step of selecting paths successively comprises selecting paths corresponding to diverse values of the output voltage.

This provides the advantage of ensuring that all possible levels of the output voltage are considered early on in the search process. This means that the majority of behaviours are explored quickly, allowing the best behaviour to be identified and therefore a near-optimal cost to also be identified. This reduces the amount of time spent searching system behaviours that are not effective in the current time step and allows an approximate path with a cost that is close to optimum to be discovered quicker, allowing for more of the search space to be discarded and reducing the computational effort required.

Preferably, the discrete-time model of the pulse system is a transformation of a continuous-time model of the pulse system.

This provides the advantage of being more computationally efficient than a continuous-time model, as a discrete time model can provide exact results for a single fixed time step which is computationally efficient.

Preferably, the discrete-time model is a discrete-time state-space model.

This provides the advantage of allowing the state of the system to be accurately tracked and modelled over each time-step.

Preferably, the control sequence is a sequence of switching states of the switches over time-steps with insertion of blanking intervals between switching of switches, and the discrete-time model of the pulse system models the insertion of the blanking intervals.

The control sequence containing blanking intervals provides the advantage of protecting the components of the switching circuit. If suitable blanking intervals are not inserted, problematic combinations of switches will be on at the same time, which can damage the circuit. The insertion of a blanking interval means that a first switch is guaranteed to be in the off state before a second switch is turned to the on state, preventing both switches from being on at the same time and damaging the circuit. The model of the pulse system including the insertion of the blanking intervals allows for a more accurate model to be provided and therefore, a more accurate solution.

Preferably, the method comprises adjusting the target profile before determining the paths of switching states for respective time-steps in a manner that restricts magnitudes of rates of change of the target profile.

In certain scenarios, the method is unable to effectively prune the search space. This occurs when the cost grows rapidly deep into the search space, meaning that the majority of the earlier branches before that point will have a lower cost, and so will not be pruned. This leads to the MPC algorithm requiring a large amount of computational effort to arrive at the optimal path, as more of a path must be explored before it is discarded. The advantage of performing the above mentioned adjustment is that, by restricting the magnitude of rate of change, the cost will rise closer to its final cost earlier in the path. This means that non-optimal paths can be identified and discarded earlier, leading to the optimal path being with less computational effort.

Preferably, the target profile is a target profile of current through the pulse coil, and the electrical state of the pulse system predicted by the discrete-time model includes a current through the pulse coil.

This has the advantage of allowing the system to accurately model the effects that the target profile will have on the system.

Preferably, the step of adjusting the target profile comprises: predicting the output voltage at each time-step from the target profile using a model of the transfer of energy stored in the at least one capacitive discharge element to the reactive circuit, and applying a predetermined limit to the change in current in each time-step that is equal to the product of the length of the time-step and the quotient of the predicted output voltage at each time step divided by the inductance of the reactive circuit, or a predetermined fraction of that product.

This has the advantage of ensuring that the target profile does not include a step-change that would lead to a rate-of-change greater than the theoretical maximum that the circuit can achieve, allowing for the cost increase to be earlier in the search space, leading to the optimal path being identified with less computational effort.

Preferably, the step of adjusting the target profile comprises applying a predetermined limit to the change in current in each time-step that is equal to the product of the length of the time-step and the quotient of the initial output voltage divided by the inductance of the reactive circuit, or a predetermined fraction of that product.

This provides the advantage of allowing for the first rate-limited reference calculated to be iteratively improved, to provide a second rate-limited reference that takes into account the change in voltage due to the at least one capacitive discharge element discharging over time. This leads to a more accurate value for the rate-of-change.

Preferably, the reactive circuit further comprises a filter circuit arranged to filter the input to the reactive circuit before application to the pulse coil.

This provides the advantage of allowing undesirable high-frequency components of the voltage applied to the pulse coil to be removed.

The present invention is applicable to any type of reactive circuit including a pulse coil for generating electromagnetic pulses. It has particular application to a reactive circuit that is a transcranial magnetic stimulation (TMS) circuit, in which case the electromagnetic pulses are transcranial magnetic stimulation pulses. Other applications include pulse power systems for particle accelerators, fusion reactors, magnetic launch systems, etc.

The switching circuit may comprise plural capacitive discharge elements and the output voltage is a multi-level output voltage. This is advantageous in many applications where it is desired to provide fine control across a wide voltage range that is greater than a typical voltage across a single capacitive discharge element.

The switching circuit may comprise plural switching modules, each switching module comprising an comprising a capacitive discharge element and switches arranged to connect a capacitive discharge element across a module output, the switching module outputs being cascaded to provide the multi-level output voltage.

Such a cascade of switching modules is a convenient way to structure the switching circuit, as the individual switching modules may each be controlled and operated in a similar manner. For example, switching state of the switching modules may represent the switching states of the switches within the respective switching modules.

Preferably, the switches in each switching module may be arranged in a bridge arrangement.

This provides the advantage of allowing the switching modules to take on and switch between multiple different states.

Preferably, the switching modules have switching states including a forward switching state connecting the capacitive discharge element across the module output in a forward direction, a reverse switching state connecting the capacitive discharge element across the module output in a reverse direction, and at least one bypass switching state not connecting the module output across the module output.

This provides the advantage that each module has flexibility in the switching states that it can take on, allowing a variety of values that the switching circuit can output.

According to a second aspect of the present invention, there is provided a method of operating a pulse system comprising a reactive circuit including a pulse coil for generating electromagnetic pulses, and a switching circuit comprising plural switches arranged to switchably connect at least one capacitive discharge element to provide an output voltage as an input to the reactive circuit, the method comprising: deriving an optimised control sequence for the pulse system that is a sequence of switching states of the switches over time-steps by a method according to the first aspect of the present invention; and operating the pulse system under the control of the derived, optimised control sequence.

According to further aspects of the present invention, there is provided a computer program capable of execution by a computer apparatus and configured, on execution, to cause the computer apparatus to perform a method according to the first aspect, a computer-readable storage medium storing such a computer program, and a computer apparatus configured to perform a method according to the first aspect.

Embodiments of the invention will now be described, by way of non-limitative example, with reference to the accompanying drawings in which:

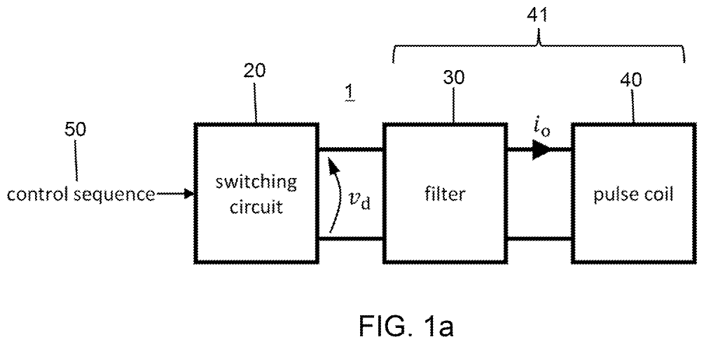

FIG. 1a shows a pulse system;

FIG. 1b shows an example implementation of the different blocks shown in the generic pulse system;

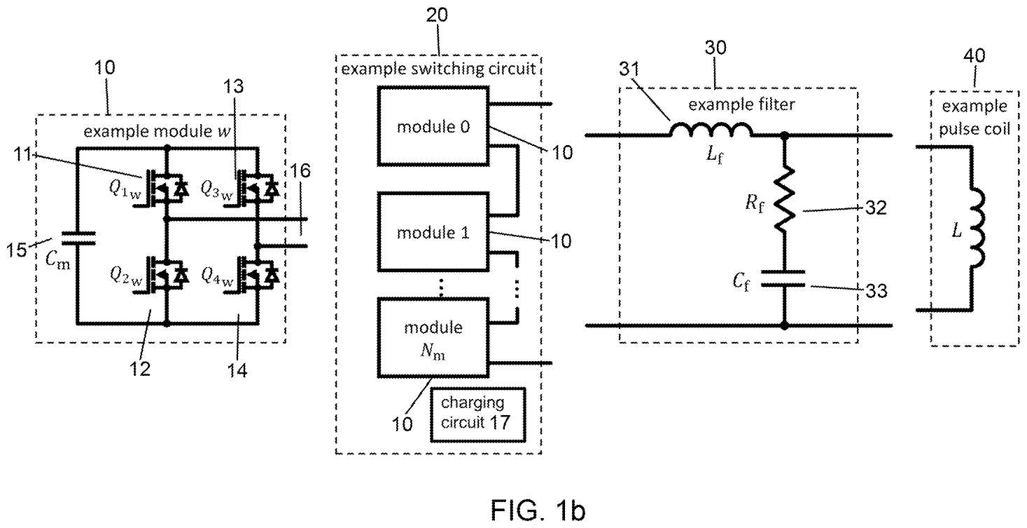

FIG. 2 shows an example of the output pulse current of the pulse system whose control sequence has been generated by the MPC algorithm, as well as an example of the control sequence and the voltage at different points in the circuit;

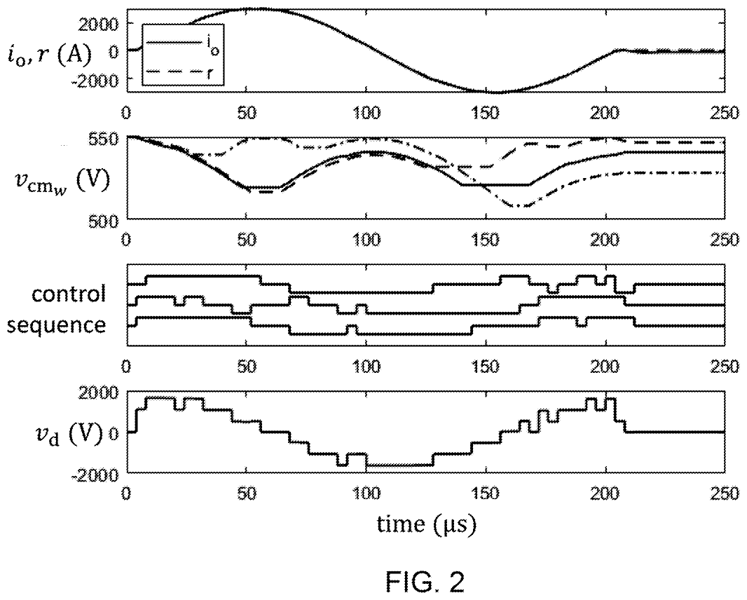

FIG. 3 shows a high level flowchart of the method for deriving an optimised control sequence for the pulse system using MPC;

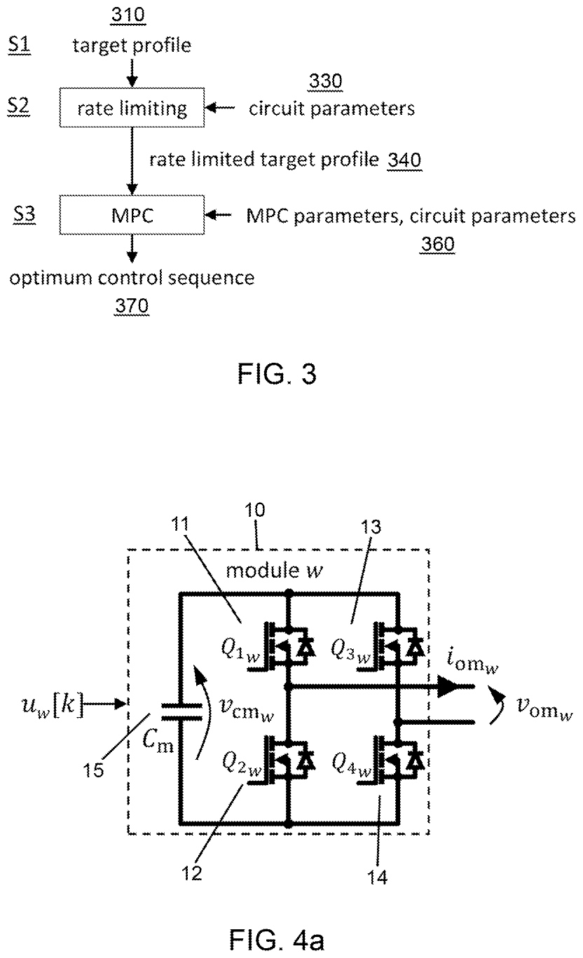

FIG. 4a shows one embodiment of a switching module used in the pulse system;

FIG. 4b shows one embodiment of the pulse system;

FIG. 4c shows a table illustrating the different switching states of the pulse system;

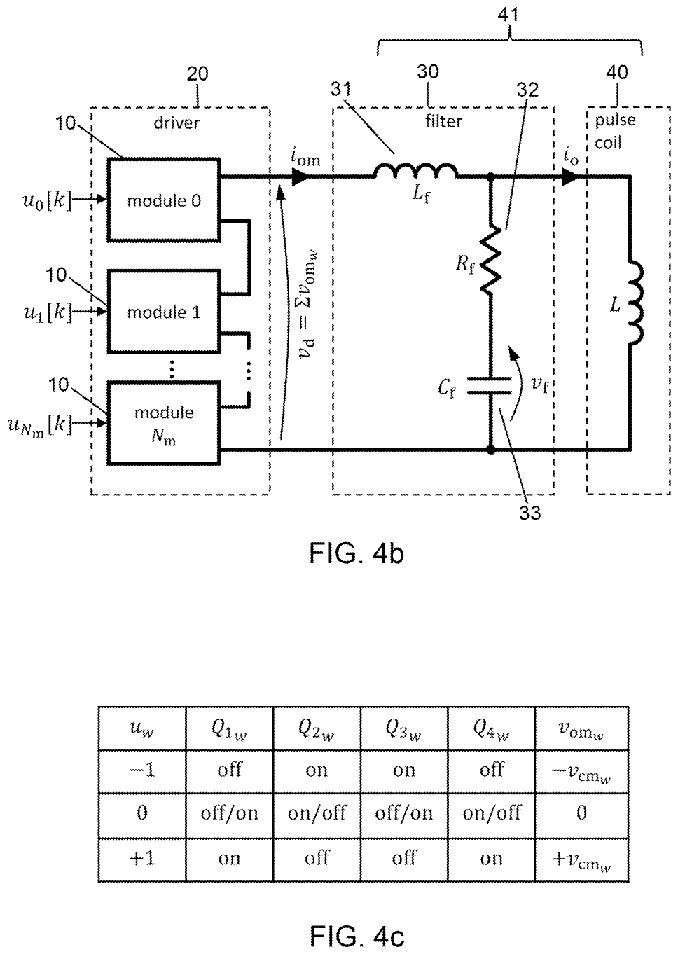

FIGS. 5a, 5b, and 5c shows three circuit diagrams for the three states of the pulse system when n is greater than 1, equal to 0, and equal to −1;

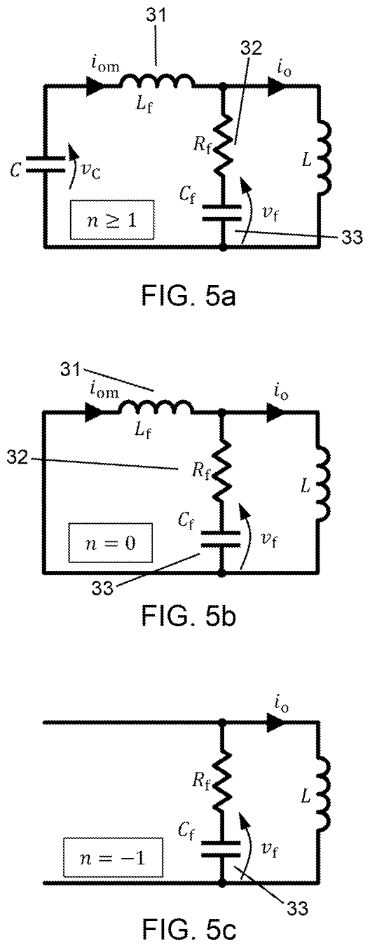

FIG. 6a shows one embodiment of a switching module used in the pulse system;

FIG. 6b shows waveforms illustrating the blanking interval effect on circuit modelling;

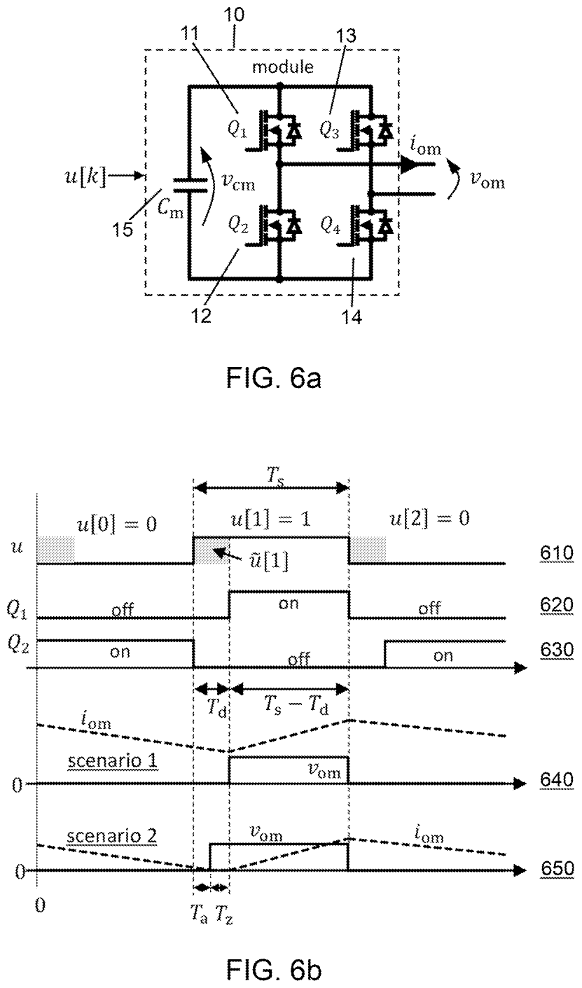

FIG. 7 shows a flowchart of the stages involved in the step( ) function;

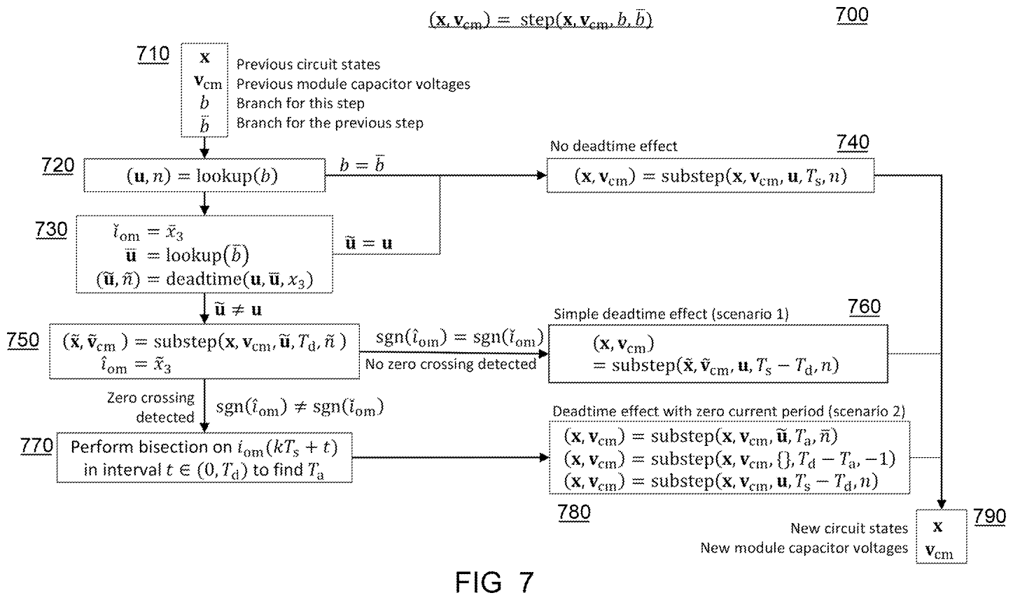

FIG. 8 shows a flowchart of the stages involved in the substep( ) function;

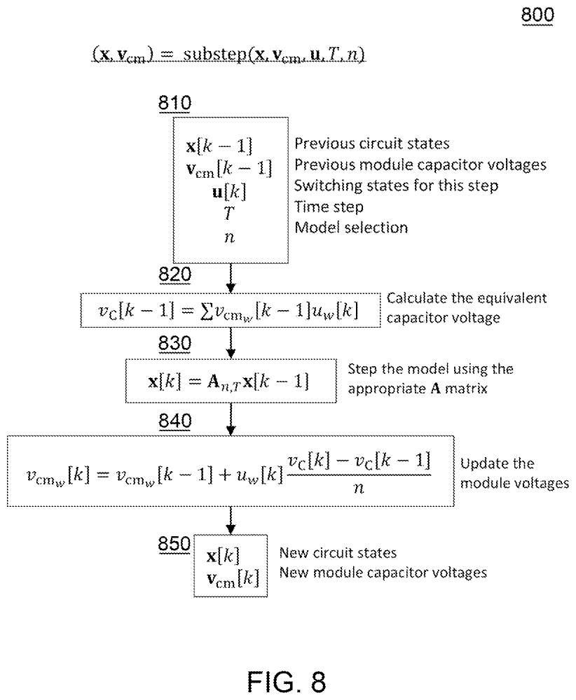

FIG. 9 shows a flowchart of the stages involved in the mpc( ) function;

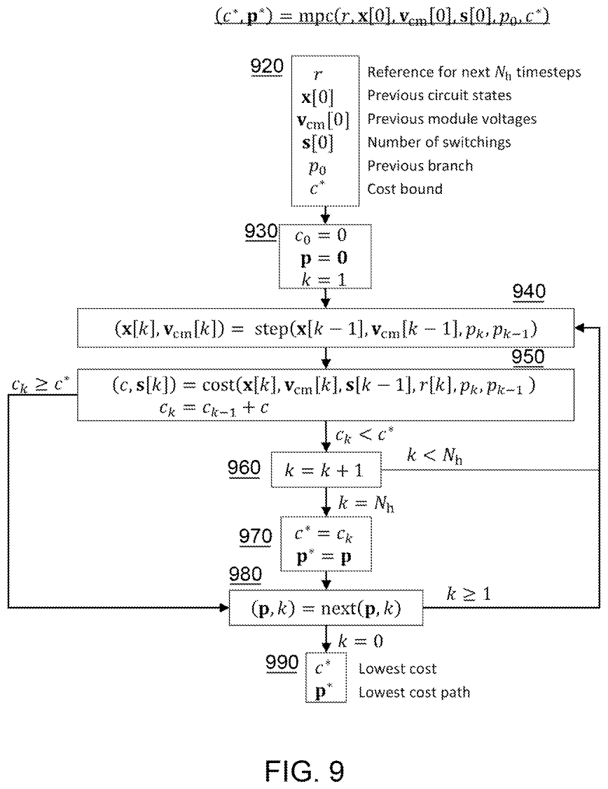

FIG. 10 shows an example tree of search paths for a one-module system with a window length of three;

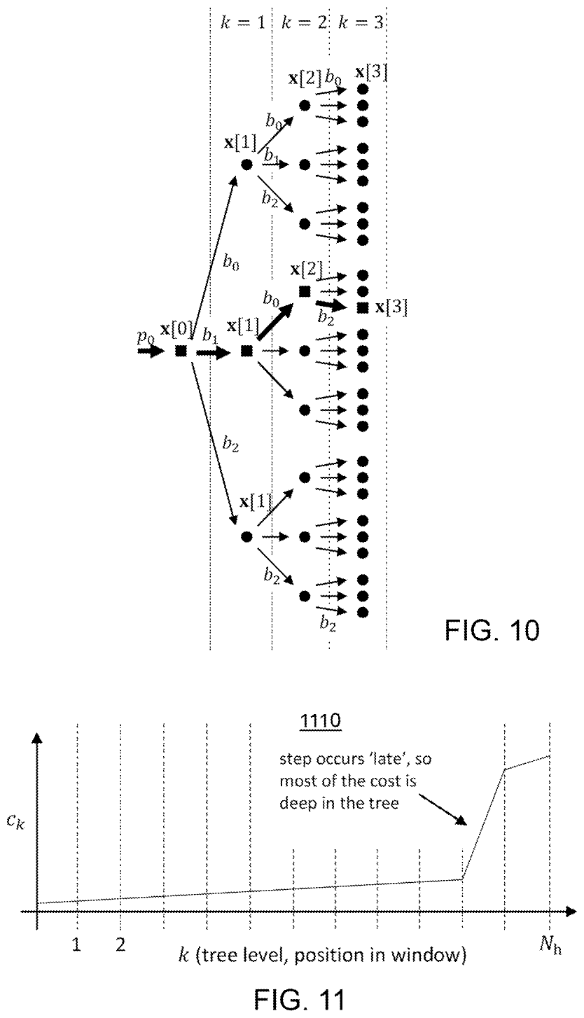

FIG. 11 shows an example of the effects of step-change of target profile deep in the search tree; and

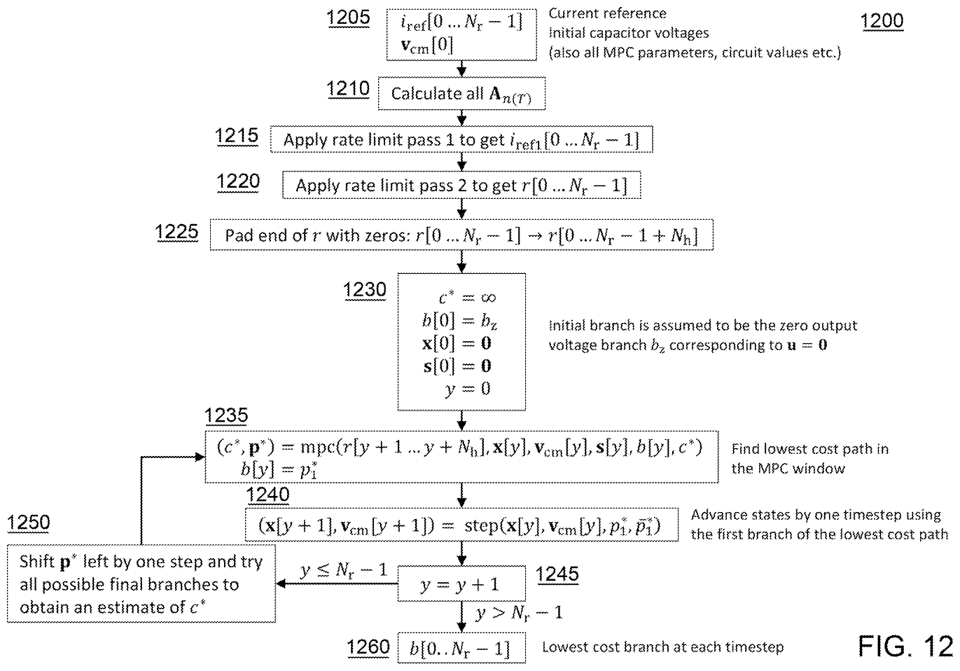

FIG. 12 shows a flowchart of the stages involved in the overall algorithm.

FIG. 1a shows a pulse system 1 comprising a switching circuit 20, a filter circuit 30 and a pulse coil 40. These components of the pulse system 1 are arranged as follows, with reference to FIG. 1b which shows an implementation of each of the blocks shown in FIG. 1a.

The pulse coil 40, represented by an inductance L in FIG. 1b, generates electromagnetic pulses as a result of the current iₒ flowing through the pulse coil 40.

The filter circuit 30 is arranged to filter an input having an input voltage v_{d} before application to the pulse coil 40. In the example of FIG. 1b, the filter circuit 30 takes the form of a damped second order low pass filter circuit comprising i) an inductance L_{f} 31 in series with the pulse coil 40, and ii) a series arrangement of a capacitance C_{f} 33, and a damping resistor R_{f} 32 in parallel with the pulse coil 40. However, this form of the filter circuit 30 is not limitative and in general the filter circuit 30 may take any suitable form.

The filter circuit 30 and the pulse coil 30 together form a reactive circuit 41 to which the input having the input voltage v_{d} is applied. The reactive circuit 41 may be a transcranial magnetic stimulation (TMS) circuit, in which the pulse coil 40 generates electromagnetic pulses that are TMS pulses. In that case, the pulse coil 40 and the filter circuit 30 may be optimised to generate TMS pulses for clinical applications of TMS. However, the techniques described herein may be applied more generally to any other type of reactive circuit, for example reactive circuits in particle accelerators, fusion reactors, magnetic launch systems, etc.

The switching circuit 20 drives the reactive circuit 41 by generating a multi-level output voltage of the switching circuit 20 that is supplied as the input to the reactive circuit 41. The switching circuit 20 comprises a plurality of switching modules 10. In this example, three switching modules 10 are shown by way of illustration in FIG. 1b, but in general any number of switching modules 10 may be provided.

Each switching module 10 has the arrangement shown in FIG. 1b as will now be described. Each switching module 10 comprises a capacitive discharge element 15 and a bridge arrangement of switching elements 11-14 that can switchably connect the capacitive discharge element 15 across the module output 16 of the switching module 10. The switching elements 11-14 act as switches. In this example, the switching elements 11-14 are shown by way of illustration in FIG. 1b as being N-Channel MOSFET (metal oxide semiconductor field-effect transistor) devices including a body diode, but in general the switching elements 11-14 may comprise semiconductor switches of any suitable type, for example alternatively being an insulated gate bipolar transistor (IGBT). Similarly, the individual switching elements 11-14 may be implemented by plural parallel switches in order to increase its effective current carrying capacity.

In this example, the bridge arrangement of switching elements 11-14 is a full-bridge (or H-bridge) by way of illustration, but in general other bridge arrangements of switches may be applied.

Each capacitive discharge element 15 is also connected to a capacitor charging circuit 17 that charges the capacitive discharge element 15 in between pulses or pulse-groups.

The arrangement of the switching modules 10 is a cascade in which the module outputs 16 are cascaded to provide the output voltage of the switching circuit 20 that is supplied as the input to the reactive circuit 41 as a multi-level voltage. In this example, the cascade of the switching modules 10 is shown by way of illustration in FIG. 1b as a simple cascade in which the module outputs 16 are connected in series, but in general any cascade arrangement of the switching modules 10 may be applied.

In general, the multi-level output voltage of the switching circuit 20 may be a unipolar or bipolar output voltage, as is known for cascaded switching modules 10.

As a result of the bridge arrangement of switching elements 11-14, the switching modules 10 each have switching states representing the switching states of the individual switching elements 11-14 in the respective switching module 10. That is, each switching state of the switching module 10 corresponding to a different combination of switching states of the switching elements 11-14. In this example, the switching states of the switching modules 10 include a forward switching state in which the switching states of the switching elements 11-14 cause the switching elements 11-14 to connect the capacitive discharge element 15 across the module output 16 in a forward direction, a reverse switching state in which the switching states of the switching elements 11-14 cause the switching elements 11-14 to connect the capacitive discharge element 15 to be connected across the module output 16 in a reverse direction, and at least one bypass switching state in which the switching states of the switching elements 11-14 not cause the switching elements 11-14 to connect the capacitive discharge element 15 across the module output 16.

Such an arrangement of cascaded switching modules 10 is convenient and simplifies the control and operation of the switching circuit 20 as it is possible to select and control the switching states of the switching elements 11-14 by selecting and controlling the switching states of the switching modules 10. However, in general the switching circuit 20 may have any arrangement of switches that switchably connect capacitive discharge elements to provide a multi-level output voltage.

In this example, the cascade of switching modules 10 is used to switchably connect plural capacitive discharge elements to provide a multi-level output voltage of the switching circuit 20, but even more generally the switching circuit 20 could include a single capacitive discharge element and be to provide a single level (bipolar or unipolar) output voltage.

The control sequence 50 for the pulse system 1 defines the sequence of switching states of the individual switching modules 10 over time, and hence the input to the reactive circuit 41. The control sequence 50 is therefore selected to cause the electromagnetic pulse generated by the pulse coil 40 to take the desired form, which may be represented by a target profile 310.

A conventional approach to generate the control sequence 50 is to use carrier-based pulse width modulation (PWM), where a target pulse system output voltage profile is compared with a triangular carrier signal. A high-fidelity output is achieved when the ratio of carrier signal frequency to target profile 310 signal frequency is large (20 or more), and the voltage variation of the capacitive discharge element 15 over time is negligible. For high power pulsed-load applications, the drawbacks of carrier-based PWM are significant. First, the assumption that the voltage variation of the capacitive discharge element 15 over time is negligible is usually not valid for pulsed power system; the voltage of the capacitive discharge element 15 may be allowed to fall a large amount (e.g., one half) over the pulse duration so that a small capacitance value can be used, thus reducing cost and physical size. Second, the practical limit on the maximum switching frequency of high power IGBTs is limited (˜20 kHz). For high power pulse systems where the target profile 310 frequency is in the range of 2 kHz to 5 kHz, the ratio of carrier signal frequency to target profile 310 signal frequency is smaller than the minimum recommended value of 20 for PWM. The combined effect is that there will be a significant deviation of the output pulse from the target profile 310 when a carrier-based PWM method is used to generate the control sequence 50.

The approach described herein aims to overcome these issues, and provide a method that derives a control sequence that is optimised to cause the electromagnetic pulse generated on application of the control sequence to more closely follow the target profile 310. Using an MPC approach to generate the control sequence 50 alleviates these problems by treating the generation as an optimisation problem at each time step. By taking the calculated voltages of the capacitive discharge elements 15 and other circuit states as feedback at each time step, the MPC approach determines the next switching state to generate the target output pulse. This leads to five key advantages when compared to carrier-based PWM:
1. Any circuit state can be made to follow the target profile 310; typically, the chosen state is the pulse coil 40 current. In contrast, a carrier-based PWM approach controls pulse coil 40 current indirectly via control of the voltage v_{d} produced by the switching circuit 20.
2. Non-ideal operation of the pulse system 1 is inherently compensated for; for example, the effect of blanking interval and forward voltage drop of the switching elements 11, 12, 13, 14 in the switching circuit 20. This is very difficult to achieve with a carrier-based PWM approach.
3. Variation in capacitive discharge element 15 voltage over the duration of a pulse is inherently compensated for. In contrast, a carrier-based PWM approach requires additional feedback systems to compensate for variation in the voltage of the capacitive discharge element 15.
4. It is conceptually simple to extend operation to complex systems, such as systems that contain a filter circuit 30 between the switching circuit 20 and the pulse coil 40, using the MPC framework. This means that, for example, pulse coil 40 current can be made to follow the target profile 310 accurately, despite the action of a filter circuit 30. This is very difficult to achieve with a carrier-based PWM approach.
5. Fewer switchings are required to achieve a given quality of the output (deviation of the chosen circuit state from the target profile 310) compared to that required by a carrier-based PWM approach.

However, MPC is significantly more computationally expensive than carrier-based PWM. This is due to the use of optimisation, as a large search space of possible solutions exist. These solutions all have to be considered and searched to find the optimal control sequence 50, which requires a considerable amount of computational effort. This makes an online approach difficult to apply. While conceptually possible, in practice limits on available computational power make the method difficult to perform, resulting in practice in the following of the target profile being inaccurate.

However, it has been appreciated that an MPC approach can be applied offline, because pulsed power applications, such as TMS, are characterised by short, discrete, and non-periodic outputs. Accordingly, in the techniques described herein, the control sequence is derived offline, prior to operating the pulse system 1 under the control of the derived, optimised control sequence. Offline MPC precomputes the control sequence 50 required to generate a target output pulse. When a pulse is to be produced by the pulse system 1, the appropriate control sequence 50 is selected from the precomputed library and fed to the pulse system 1. The method may be performed as follows.

To assist understanding, reference is first made to FIG. 2, which shows plots on the same timeline for an example of the derivation of an electromagnetic pulse from a target profile. The first plot shows the target profile r (being a current in this example) and the output current iₒ of electromagnetic pulse generated by the pulse system 1 whose control sequence has been generated by the MPC algorithm. The second plot shows the voltages v_{cm}_{_{w}} across the capacitive discharge elements 15 of the switching modules 10. The third plot shows the control sequence 50 that is derived, which comprises a varying switching state for each of the switching modules 10. The fourth plot shows the resultant input voltage v_{d} of the input to the reactive circuit 41. Evidently, the output pulse current follows its target profile throughout the whole pulse duration even though the voltages of the capacitive discharge elements 15 decrease over time.

A method of deriving an optimised control sequence 50 for the Pulse system 1 is shown in FIG. 3 and will now be described in detail.

FIG. 3 illustrates a method including steps performed in a computer apparatus. In these drawings, the steps of the method are performed in functional blocks of the computer apparatus. The functional blocks process data representing various information described in detail below.

The computer apparatus may be implemented as a computer apparatus executing a computer program. In this case, the computer program is capable of execution by the computer apparatus and is configured, on execution, to cause the computer apparatus to perform the method including the steps of the functional blocks. Such a computer apparatus may be any type of computer system but is typically of conventional construction. The computer program may be written in any suitable programming language.

The computer program may be stored on a computer-readable storage medium, which may be of any type, for example: a recording medium which is insertable into a drive of the computing system and which may store information magnetically, optically, or opto-magnetically; a fixed recording medium of the computer system such as a hard drive; or a computer memory.

The method as shown in FIG. 3 has three steps as follows.

In step S1, a target profile 310 is received for an electromagnetic pulse to be delivered by the pulse coil 40. The method also makes use of circuit parameters 330 (e.g., various inductance, capacitance, resistance values, and initial voltages and currents, blanking time, etc.) and MPC parameters 360 (e.g., length of the window, cost weightages, etc.). However, these parameters need to be received only once, and need not be re-entered every time a new target profile 310 is provided.

In step S2, the target profile 310 is adjusted, if required, before determining the paths of switching states for respective time-steps in a manner that restricts magnitudes of rates of change of the target profile 310. This step will be described in detail below.

In step S3, the MPC algorithm determines the optimum control sequence 370 which is a sequence of switching states over successive time-steps across the period of operation corresponding to the target profile 310.

The method is performed offline prior to operating the pulse system 1 under the control of the derived, optimised control sequence 370. This means that the control sequence can be prepared in advance and the pulse system 1 is not impacted by the time taken to arrive at the optimised control sequence.

In the example described in more detail below, the target profile 310 is a target profile 310 of current through the pulse coil 40. More generally, the target profile 310 could be a target profile 310 for any electrical parameters of the electromagnetic pulse.

The method of determining the control sequence 50 performed in step S3 will now be discussed in detail.

For reference, parameters of the electrical state of the pulse system 1 used in the description below will now be described with reference to FIGS. 4a to 4c. In the pulse system 1, the four switching elements 11-14 of each switching module 10 are labelled Q₁_{_{w}} to Q₄_{_{w}}, and the energy storing capacitive discharge elements 15 have a capacitance Cₘ and a voltage v_{cm}_{_{w}} as shown in FIG. 4a. A constraint on the switching module 10 is that it cannot have both the left-hand switching elements 11, 12 and/or both the right-hand switching elements 13, 14 switched on at the same time; this avoids a short circuit across the capacitive discharge element 15. As a result, in this example the switching state u_{w} of the switching module 10 has three possible values that reflect the three possible states of its output voltage vₒₘ_{_{w}}. These three states are shown also in FIG. 4c. In this example, the output voltage v_{d} of the switching circuit 20, which is the input to the reactive circuit 41, is the sum of these output voltages vₒₘ_{_{w}}.

As shown in FIG. 4b, for a system with Nₘ switching modules 10 a switching vector

$$u = [ \begin{matrix}{u}_{0} \\ {u}_{1} \\ ⋯ \\ {u}_{{N}_{m}}\end{matrix} ]$$

can be defined, which can take N_{b}=3^{N}^{ₘ} distinct combinations, due to the three unique states that each switching module 10 can transition between. Thus, a switching matrix U of size Nₘ×N_{b} can be defined, each column of which is a switching vector termed as a ‘branch’ bⱼ, j∈{0 . . . N_{b}−1}. This switching matrix stores all possible branches for that number of switching modules 10. For example, where three switching modules 10 are present, the switching matrix size will be 3×27, where the total number of switching vectors or ‘branches’ is 27 and each branch has 3 elements, representing the 3 states of the switching modules 10. The following pseudocode is used to select a particular branch using a function called lookup( ).

$$( u , n ) = lookup ( b )$$

$$u = {U}_{i , b + 1}$$

$$n = \sum \vert {u}_{w} \vert for w ∈ \{ 0 … {N}_{m} - 1 \}$$

The task of the MPC algorithm is to determine the optimal sequence of branches b[y] (and therefore u[y], where u=[u₀ . . . u_{N}_{ₘ}₋₁]) to cause the output current iₒ[y] through the pulse coil 40 to follow a user-defined target profile 310 i_{ref}[y], whilst limiting the number of switching instances of the switching modules 10, and the voltage deviations of the capacitive discharge elements 15, among other constraints. Here, y∈{0 . . . Nᵣ−1}, and Nᵣ is the number of samples in the target profile 310.

The determination of the control sequence 50 uses a discrete-time model of the pulse system to predict an electrical state of the pulse system 1. The electrical state comprises the current iₒ through the pulse coil 40, a voltage v_{cm}_{ᵥ} of the capacitive discharge element 15 in respect of each switching module 10, and other electrical parameters of the pulse system 1 that are variable in operation, for example currents and voltages in respect of components of the pulse system 1.

An example of the discrete-time model will now be described, with reference to FIGS. 5a-5c, which show equivalent circuit diagrams or the circuit states of the pulse system 1. In this example, the discrete-time model is a discrete-time, state-space model, but in general any other type of discrete-time model could be used.

The discrete-time model of the pulse system 1 is a transformation of a continuous-time model of the pulse system 1. Mathematically,

$$x [ k ] = {A}_{n , T} x [ k - 1 ]$$

$$where$$

$${A}_{n , T} = {e}^{T {\tilde{A}}_{n}} ,$$

T is the step time. Ãₙ for n≥1 is the continuous-time linear state transition matrix of the circuit, as shown in FIG. 5a, where the switching circuit 20 has been replaced with a single equivalent capacitor C:

$$C [ k ] = \frac{{C}_{m}}{n} , n = \sum \vert {u}_{w} \vert for w ∈ \{ 0 … {N}_{m} - 1 \} ,$$

where n is the number of switching modules 10 that are in a non-zero state. T is the sampling time for the continuous-to-discrete transformation and k indicates the k-th time-step of the discrete-time model of the system.

The circuit state vector x includes the current iₒ through the pulse coil 40, the voltage v_{f} across the capacitor of the filter circuit 20, output current iₒₘ of the switching circuit 20, and the voltage v_{C} across the equivalent capacitor C corresponding to the switching circuit 20, which is derived as the sum of the output voltage vₒₘ_{_{w}} of the of the modules 10, which are themselves derived from the voltages v_{cm}_{_{w}} across the capacitive discharge element 15 of each switching module 10.

$$x = [ \begin{matrix}{i}_{o} \\ {v}_{f} \\ {i}_{o m} \\ {v}_{C}\end{matrix} ] for n \geq 1$$

There are also A_{0,T} and A_{-1,T} models, corresponding to ‘all switching modules 10 in zero state’ as shown in FIG. 5b, and ‘switching modules 10 in blanking interval state’ as shown in FIG. 5c, respectively. These are reduced-order models:

$$x = [ \begin{matrix}{i}_{o} \\ {v}_{f} \\ {i}_{m}\end{matrix} ] for n = 0$$

$$= [ \begin{matrix}{i}_{o} \\ {v}_{f}\end{matrix} ] for n = - 1$$

For n≤0, the output voltages vₒₘ_{_{w}} of the switching modules 10 are preserved (v_{C}[k]=v_{C}[k−1]).

For n=−1 the output current iₒₘ of the switching modules 10 is held at zero (this can occur during the blanking interval).

If T is constant (e.g., T=Tₛ, where Tₛ is a fixed sample time) then A_{n,T} is constant. This means that the A_{n,T} matrices can be precomputed at start-up of the algorithm (there is no need to recompute at each step). This is advantageous as the matrix exponential is expensive to compute.

However, the model described above does not capture the blanking interval effect of the switching circuit 20. Inserting a blanking interval between the switching of switches in each leg of the switching module 10, and is required to avoid potentially damaging the switching module 10. This blanking interval is illustrated in the second and third waveforms of FIG. 6b discussed below. It causes the output voltage vₒₘ_{_{w}} of the switching module 10 to deviate from the ideal case. It causes a possible dependency of output voltage vₒₘ_{_{w}} of the switching module 10 on the instantaneous output current of the switching module 10. Depending on whether the output current iₒₘ of the switching module 10 crosses zero during the blanking interval, there can be two possible scenarios, described below.

The circuit modelling of the blanking interval effect will now be described with reference to FIGS. 6a and 6b. FIG. 6a shows an individual switching module 10 and corresponds to FIG. 4a. FIG. 6b shows waveforms associated with the switching module 10, wherein the first waveform is the switching state u, the second and third waveforms are the states of the left hand switching elements 11 and 12, labelled Q₁ and Q₂, and the fourth of and fifth waveforms are the output current iₒₘ and output voltage vₒₘ of the switching module 10 in scenarios 1 and 2 discussed below.

Scenario 1 is as follows. The output current iₒₘ of the switching module 10 does not fall to zero during the blanking interval, so managing the blanking interval is simple: break the step into two consecutive substeps, one of length T_{d} and the other one of length Tᵣ=Tₛ−T_{d}. Accordingly, this requires two sets of state-space matrices, A_{n,T}_{_{d}} and A_{n,T}_{ᵣ}.

Scenario 2 is as follows.

The output current iₒₘ of the switching module 10 falls to zero during the blanking interval. The time at which it hits zero depends on circuit behaviour and can take any value T_{d}∈(0, T_{d}). The following two tasks must be performed in this scenario:
1. Find Tₐ, e.g., using a bisection approach on iₒₘ(t) in the time interval t∈(kTₛ, kTₛ+T_{d}).
2. Break the step into three consecutive substeps of Tₐ, T_{z}=T_{d}−Tₐ and Tᵣ=Tₛ−T_{d}.

This requires either:
1. the direct computation of the matrix exponentials A_{n,T}_{ₐ} and A_{n,T}_{_{z}} for a given step which is computationally very expensive as it needs to be done for every bisection step, or,
2. the pre-computation of A_{n,gT}_{_{d}}_{/N}_{_{g}} where g∈{1 . . . N_{g}} with a high enough N_{g} to make a good approximation of the time Tₐ. This means that all A matrices can be precomputed, which speeds up operation very considerably.

The following pseudocode models the insertion of the blanking intervals in to the discrete-time model of the pulse system 1 using a function called deadtime( ).

```
(ü, n) = deadtime(u, ū, iₒₘ)
for w ∈ {0 ... Nₘ − 1}
  if (u_{w} > ū_{w} and iₒₘ > 0) or (u_{w} < ū_{w} and iₒₘ < 0)
  then
    ü_{w} = ū_{w}
  else
    ü_{w} = u_{w}
n = Σ|ü_{w}|
```

A method of updating the electrical parameters over a single time-step is described here that considers the different scenarios described above. The method comprises two functions called step( ) and substep( ) as shown in FIGS. 7 and 8, respectively. The step( ) function determines what blanking interval effect scenario is experienced by a switching module 10 at the current time step using the lookup( ) and deadtime( ) functions 730 described previously, and based on this decision, the substep( ) function is called 750 with appropriate input arguments if it is determined that there is a blanking interval effect.

The substep( ) function takes the previous circuit states, the previous capacitor voltages of the switching modules 10, the switching states for this time step, the time step duration, and the number of switching modules 10 in a non-zero state as the inputs in step 810; and returns the new circuit states and the new capacitor voltages of the switching modules 10 as the outputs in step 850. It starts by calculating the voltage of the equivalent capacitor of the switching circuit 20 of the previous time step in step 820, and then steps the circuit model into the current time step using the appropriate A_{n,T} matrix to update the circuit states in step 830. Similarly, the capacitor voltages of the switching modules 10 are updated using the new switching states in step 840. Finally, the step( ) function outputs the new circuit states and the new capacitor voltages of the switching modules 10 at the k-th time step in step 850.

The step( ) method shown in FIG. 7 may be repeated over successive time-steps to predict the electrical state of the pulse system 1 in an iterative manner over the period of the target profile 310.

The cost function will now be discussed.

The determination of the control sequence 50 uses the electrical state of the pulse system 1 predicted by the discrete-time model to determine an optimal path of switching states over a window of time-steps. In particular, the optimal path is the path through switching states of the switching modules 10 over a series of time-steps that minimises a cost function. The cost function is selected to optimise the desired properties of the control sequence 50 determined for a given target profile 310. The cost function provides a value representing how costly the current path is, with this value allowing the possible paths to be compared to each other. The value is calculated based on the desired behaviour of the circuit and/or the predicted behaviour of the system.

The cost function may comprise the following cost contributions. These cost contributions are defined for any particular path through switching states of the switching modules 10. For any path, each cost contribution will take a value at each time-step, which may be summed to derive the total value of the cost contribution. This cost can then be used to evaluate how costly a particular path is compared to other possible paths.

The cost contributions may be functions of the target profile 310 and/or variables which are dependent on the path.

Such variables may include a variable representing an electrical parameter of the electrical state of the pulse system 1 along the path of switching states. For example, such a variable may represent the voltages of the capacitive discharge elements 15 of the switching module 10.

Such variables may include a variable representing the switching states of the switching modules 10, for example the value of u_{w}.

Where plural cost contributions are used, for each path the cost contributions may be summed (or more generally combined in some other way) to provide a total value of the cost function, which may also be referred to as the “cost” of the path.

In all cases, the cost function has a deviation cost contribution representing deviation of an electrical state of the pulse system 1 predicted by the discrete-time model from the target profile 310 over the window of time-steps. This causes the optimisation to find a control sequence 50 that tends to reduce such deviation.

For example, for the TMS application, the cost contribution for deviation of the pulse coil 40 current iₒ from its reference r is defined as

$${c}_{r} = {{α}_{r} ( r - {i}_{o} )}^{2}$$

where αᵣ is the weightage of this cost contribution.

In addition, the cost function may have further cost contributions that represent penalties for undesirable operational characteristics of the switching modules 10. Such further cost contributions may optimise the performance of the pulse system 1, or protect the system's components from damage and unnecessary stress. This causes the optimisation to find a control sequence 50 that tends to improve the operational characteristics. Some examples are as follows.

The cost function may include a further cost contribution that is a voltage cost contribution that is dependent on the voltages of the capacitive discharge elements 15 of the switching modules 10. Such a voltage cost contribution may take a prohibitive value when the voltage of a capacitive discharge element 15 exceeds an overcharge threshold, and may otherwise represent a penalty for variation between the voltages of the capacitive discharge elements 15 of the switching module 10 over the window. This encourages the optimisation to prefer a control sequence 50 that has a similar voltage value for all of the capacitive discharge elements 15, and prevents a value that would damage the capacitive discharge elements 15.

As an example, for the TMS application, the cost contribution for capacitor voltage overcharge and variation can be defined as

$${c}_{v} = \{ \begin{matrix}∞ if max ( {v}_{cm} ) > {V}_{cm , max} \\ {{α}_{v} ( max ( {v}_{cm} ) - min ( {v}_{cm} ) )}^{2} otherwise\end{matrix}$$

where αᵥ is the weightage of this cost contribution, v_{cm} is the module capacitor voltage vector, and v_{cm,max} is the maximum allowed voltage for a capacitive discharge element 15. This prevents over-voltage failure of capacitors and ensures that the switching modules 10 of the pulse system 1 are not unequally stressed.

The cost function may include a further cost contribution that is at least one switching state cost contribution that is dependent on the switching states of the switching modules 10. Some examples of switching state cost contributions are as follows.

In one type of example, the switching state cost contribution may include at least one switching action cost contribution representing penalties for undesirable switching actions. This encourages the optimisation to prefer making desirable switching actions, which will increase the performance of the system and prevent potential damage and reduce stress to the circuit components that could be caused by repeatedly making undesirable switches. Some examples of switching action cost contributions are as follows.

One example of a switching action cost contribution for the pulse system 1 is a switching action cost contribution representing a penalty for switching states connecting the capacitive discharge element 15 across the output of the switching module 10, defined as

$${c}_{0} = {α}_{0} \sum \vert {u}_{w} \vert$$

where α₀ is the weightage of this cost contribution. It pushes the switching modules 10 to default to the zero-state, minimising stress on the switching modules 10. This is advantageous, as it causes the optimisation to prefer being in the configuration where two switching modules 10 are in the zero-state, compared to the situation where two switching modules 10 are in opposite, opposing states. This prevents undue stress from being placed on the capacitive discharge elements 15.

Another example of a switching action cost contribution for the TMS system is a switching action cost contribution representing a penalty for simultaneous change of the switching state of plural switching modules 10, defined as

$${c}_{q} = {α}_{q} \sum \vert {u}_{w} - {\overline{u}}_{w} \vert$$

where α_{q} is the weightage of this cost contribution. Including this in the cost minimises the switching stress on the switching modules 10 of the TMS system, as it places a cost on unnecessary switches, discouraging them from happening.

Another example of a switching action cost contribution for the TMS system is a switching action cost contribution representing a penalty for changes in the output voltage at each time-step, defined as

$${c}_{δ} = {α}_{δ} \sum ( {u}_{w} - {\overline{u}}_{w} )$$

where α_{δ} is the weightage of this cost contribution. Including this in the cost minimises large step-changes in output voltage in a single time-step which will cause greater stress on the filter circuit 20 and may cause EMC problems.

In another type of example, the switching state cost contribution may include an equalisation cost contribution representing a penalty for unequal switching counts of different switching modules 10.

For example, for the TMS system, using switching count vector s, the switching modules 10 that have switched the most number of times can be identified

$${β}_{w} = \{ \begin{matrix}\begin{matrix}1 & if\end{matrix} & {s}_{w} = max ( s ) \\ 0 & otherwise\end{matrix}$$

At any time-step, the switching module(s) 10 that has switched the most times so far will have β_{w}=1 for w∈{0 . . . Nₘ−1}. If this switching module(s) 10 is switched at this step, the following penalty is incurred

$${c}_{s} = {α}_{s} \sum {β}_{w} \vert {u}_{w} - {\overline{u}}_{w} \vert$$

where αₛ is the weightage of this cost contribution. Including this in the cost ensures that the switching modules 10 of the pulse system 1 are not unequally stressed, by encouraging switching to be evenly spread over all available switching modules 10, instead of repeatedly switching a single switching module 10.

The following pseudocode shows how the total cost contribution c is derived using a function called cost( ). This function involves summing all the selected cost contributions. Although the below function includes a specific combination of cost contributions, any possible combination of the mentioned cost contributions could be used.

```
(c, s) = cost(x, v_{cm}, s, r, b, b̅)
u = lookup(b)
ū = lookup(b̅)
c = cᵣ + cᵥ + c₀ + c_{q} + c_{δ} + cₛ
for w ∈ {0 ... Nₘ − 1}
  if (u_{w} ≠ ū_{w})
    s_{w} = s_{w} + 1
```

The MPC function will now be discussed, with reference to FIG. 9.

The mpc( ) function takes the reference for the next Nₕ (the length of the MPC window) time-steps, the previous circuit states, the previous capacitor voltages of the switching modules 10, the switching counter, the previous branch, and the accumulated cost bound c* as the inputs, in step 920; and returns the least cost path and its corresponding cost as the outputs in step 990.

The process starts in step 930, by initializing a local cost accumulator variable cₖ to zero, the current branch p to zero, and the time-step k to 1. It then enters an iterative search loop where the circuit states and capacitor voltages of the switching modules 10 are updated using the step( ) function in step 940 and the cost associated with the step is calculated using the cost( ) function in step 950. It then increases the time-step in the window k in step 960 and returns to step 940, provided that k is still less than the length of the window.

For respective time-steps of the control sequence 50 taken in succession, it determines an optimal path of switching states over a window (defined by Nₕ) of time-steps starting with the respective time-step by performing a search of possible paths of switching states over the window by selecting paths successively.

After the evaluation of a path, the next path (or partial path) to evaluate is found using the (p, k)=next(p, k) function in step 980. This function increments the branch number at position k, if it overflows then it performs a carry to the next higher position and resets higher k positions to 0. The function returns the position k that was incremented, e.g.:

$$p = [ 101 ] , k = 3 \to p = [ 102 ] , k = 3$$

$$p = [ 102 ] , k = 3 \to p = [ 110 ] , k = 2$$

$$p = [ 122 ] , k = 2 \to p = [ 200 ] , k = 1$$

$$p = [ 222 ] , k = 3 \to p = [ 000 ] , k = 0$$

If the returned k is zero, the last path has been evaluated and so the search terminates, otherwise the next branch in the path is evaluated.

For an initially selected path, the mpc( ) function accumulates a total value of the cost function over the entirety of the path, whereas for subsequently selected paths, it accumulates a value of the cost function over successive time-steps of the path using the local cost accumulator cₖ. At each time-step before the final time-step, the local accumulated value of the cost function cₖ is compared to a global minimum value of the cost function c* accumulated over the entirety of a path previously in the search, and if the local accumulated value has reached the minimum value then the accumulation is terminated, otherwise the accumulation is continued. On finding a path of lower cost of a path (i.e. cₖ<c* and k=Nₕ), this cost and path is recorded in step 970. On completion of the search the mpc( ) function reaches step 990 and determines that the path having a minimum total value of the cost function accumulated over the entirety of the path is the optimal path. It selects the first branch of the determined path at the respective time-step as the switching state of the control sequence 50 at the respective time-step.

An example of the search tree for one switching module 10 is shown in FIG. 10. Here, x[0], v_{cm}[0] are the circuit states and capacitor voltages of the switching modules 10, respectively, at the end of the last time-step, and p₀ is the last-taken branch. The search tree shown in FIG. 10 shows all possible paths that can be made over the next three time-steps. As per the previous discussion, a branch bⱼ (where j∈[0 1 2] for a pulse system 1 comprising one switching module 10) represents the switching sequence applied, and a path is described by a sequence of branches. For example,

$$x [ 0 ] {\to }^{{b}_{1}} x [ 1 ] {\to }^{{b}_{0}} x [ 2 ] {\to }^{{b}_{2}} x [ 3 ]$$

or more compactly p=[1 0 2] as shown in FIG. 10.

The mpc( ) function evaluates the cost of all possible paths using a depth-first approach as shown below.

$$\begin{matrix}p = [ 000 ] \\ p = [ 001 ] \\ p = [ 002 ] \\ p = [ 010 ] \\ ⋮ \\ p = [ 222 ]\end{matrix}$$

When searching the full size tree, this can become computationally expensive. To overcome this, if at any point during the evaluation of a path, the local accumulated cost cₖ exceeds the global minimum cost c* found so far, the mpc( ) function does not evaluate any more sub-paths, because the cost of each step can only be zero or positive and so can only increase from that point. Thus, using the local cost accumulator cₖ and the branch increment function next( ), the mpc( ) search algorithm ‘prunes’ the tree effectively to minimise the amount of the search space that is actually explored and so reduces the computational effort. Using this technique typically allows for large portions of the search space to be pruned, leading to a very significant decrease in the time and effort spent finding the optimal solution.

The computational effort may be reduced as follows.

As the MPC algorithm is an iterative search process, the computational effort could be prohibitively high if the whole tree must be searched in each time step, forcing the algorithm to perform a large number of evaluations. The majority of paths are not likely to provide a solution near to being optimal and so will have a high cost, meaning that once the algorithm has a good approximation, these paths can be pruned. This means that fewer evaluations will be required if the algorithm can obtain a good approximation of the least accumulated cost c* early on so that large parts of the tree need not be evaluated. The following two approaches may achieve this.
1. The MPC algorithm evaluate paths that are likely to be low-cost first. For respective time-steps of the control sequence 50 subsequent to the first, the step of selecting paths successively comprises initially selecting paths that commence with the overlapping part of the optimal path that was determined for a previous time-step of the control sequence 50. As the overlapping part was previously part of the optimal path, it is likely that any path including it in the current time-step will provide a good approximation for the new optimal path, as the majority of the path will remain the same. This leaves only N_{b} evaluations to perform corresponding to the last entry in the new path. Although this does not help with the first time-step, it gives a very significant (roughly 20 times) speedup for subsequent time-steps.
2. The MPC algorithm uses a branch mapping such that at an early stage of the search, the step of selecting paths successively comprises selecting paths corresponding to diverse values of the cascaded output voltage. Thus, a whole range of possible system behaviours is explored early on to identify a near-optimal cost. This means that the behaviours that lead to a low cost path will be explored relatively quickly, as opposed to a system behaviour with a high cost being fully explored before searching any other possible system behaviours. This allows for a reasonable approximation to be discovered sooner and be used for pruning the search space. For example, for the TMS system, the mapping of b to the underlying switching matrix U is chosen such that the early members of b correspond to a diverse number of converter output voltages, e.g.,

$${v}_{C} ( b ) = {v}_{c m} [ \begin{matrix}\begin{matrix}\begin{matrix}\begin{matrix}\begin{matrix}\begin{matrix}\begin{matrix}0 & 1\end{matrix} & - 1\end{matrix} & 2\end{matrix} & - 2\end{matrix} & 3\end{matrix} & - 3\end{matrix} & ⋯\end{matrix} ]$$

Applying this approach leads to a roughly 30% reduction in run time (assuming the first approach is also implemented).

The method of adjusting the target profile 310 performed in step S2 will now be discussed in detail.

The two methods to reduce computational effort as described above, together, give excellent results for smoothly changing target profiles 310. However, the target profile 310 supplied by the user may contain large rates-of-change (e.g., a step-change) that significantly slow down the MPC algorithm as it requires a very high number of path evaluations near the step. This is because, if the cost increases very suddenly near end of the window (due to an upcoming step-change), the MPC algorithm cannot ‘prune’ the tree effectively, as the accumulated cost will only become greater than the global minimum cost towards the end of the tree. Even if much of the shallow parts of the tree have a lower cost, the sudden increase in cost near the end of the window makes the accumulated cost c* large, so the algorithm must search deeply before it can discard a path and its corresponding sub-paths. This means that the majority of the tree will be searched and the pruning technique will have a limited effect on the computational effort required. The problem is illustrated in FIG. 11, which shows the cost against the tree level, illustrating in particular an example of such a case where a significant step change in the cost occurs deep in the tree, so most of the cost is deep in the tree.

A solution to this problem is to adjust the target profile 310 before determining the paths of switching states for respective time-steps in a manner that restricts magnitudes of rates of change of the target profile 310, so that more of the change, and the associated cost increase, happens earlier in the search tree. This means that the effect of the large rate of change is reduced and the search space can be pruned more effectively.

For example, for the TMS system, the user supplies a target profile 310 in the form of a reference sequence i_{ref} for the coil current that may have large step-changes. The method of adjusting the target profile 310 comprises applying a predetermined limit to the change in current in each time-step Δiₘₐₓ₁ that is equal to the product of the length of the time-step Tₛ and the quotient of the initial equivalent capacitor voltage v_{C}[0] divided by the sum of coil inductance L and filter inductance L_{f}, or a predetermined fraction of that product. In the sample domain, thus, a first rate-limited reference i_{ref1} is generated by applying the ‘theoretical maximum’ change in current per step of

$$Δ {i}_{max 1} [ y ] = {T}_{s} \frac{{v}_{C} [ 0 ]}{L + {L}_{f}} , for all y ∈ 0 … {N}_{r} - 1$$

This ignores the dynamics of the filter circuit 20. Here, C is the ‘equivalent capacitor’ which emulates the capacitive discharge elements 15 of all of the switching modules 10 of the switching circuit 20 in series.

$$C = \frac{{C}_{m}}{{N}_{m}} , {v}_{C} [ 0 ] = {N}_{m} {v}_{c m} [ 0 ]$$

However, the issue with the first rate-limited reference is that as the capacitive discharge elements 15 discharge, the equivalent capacitor voltage v_{C}[y] decreases. This means that the maximum achievable rate of change of coil current decreases over time. If this is not also considered, then the ‘theoretical maximum’ will become unrepresentative as the number of time-steps increases.

Under the assumption that the MPC algorithm will maintain good capacitor voltage balance, the equivalent capacitor voltage at each time-step can be predicted from the target profile 310 using a model of the transfer of energy stored in the equivalent capacitance to the pulse coil 40:

$${E}_{C} [ y ] = \frac{1}{2} C {v}_{C}^{2} [ y ] ; \qquad t = y {T}_{s}$$

$${E}_{dch} [ y ] = {T}_{s} {i}_{a v} [ y ] {v}_{coil} [ y ] = {T}_{s} {i}_{a v} [ y ] ( {Ri}_{a v} [ y ] + L \frac{1}{{T}_{s}} ( {i}_{ref 1} [ y ] - {i}_{ref 1} [ y - 1 ] ) ) = {i}_{a v} [ y ] ( {T}_{s} R {i}_{a v} [ y ] + L ( {i}_{ref 1} [ y ] - {i}_{ref 1} [ y - 1 ] ) )$$

Here,

$${i}_{a v} [ y ] = \frac{1}{2} ( {i}_{ref 1} [ y ] + {i}_{ref 1} [ y - 1 ] ) .$$

The capacitor energy and voltage decrease as:

$${E}_{C} [ y ] = {E}_{C} [ y - 1 ] - {E}_{dch} [ y ]$$

$${v}_{C} [ y ] = \sqrt{\frac{2 {E}_{C} [ y ]}{C}}$$

Thus, the method of further adjusting the target profile 310 comprises applying a predetermined limit to the change in current in each time-step Δiₘₐₓ₂ that is equal to the product of the length of the time-step Tₛ and the quotient of the predicted equivalent capacitor voltage at each time-step v_{C}[y] divided by the sum of coil inductance L and filter inductance L_{f}, or a predetermined fraction of that product.

$$Δ {i}_{max 2} [ y ] = {T}_{s} \frac{{v}_{C} [ y ]}{L + {L}_{f}}$$

This is applied to the first rate-limited reference i_{ref1} to generate a second rate-limited reference r. This is what is used as the target profile 310 for the MPC algorithm.

This second rate-limiting algorithm could be run again (and again . . . ) with i_{ref1} substituted by r to take account of any changes made to the reference in an iterative fashion. However, in practice, a single pass is all that is necessary.

Together the first and the second rate-limiting steps help in reducing computational effort by avoiding deep searches into the window. The rate-limiting process does not in any way reduce the output capabilities of the system, as the applied limit is equal to or greater than the maximum rate that the physical circuit can achieve.

The overall algorithm is shown in FIG. 12 and will now be described. It combines all three steps (S1, S2, and S3) as shown in FIG. 3 and discussed above.

The algorithm starts in step 1205 with receiving inputs from the user in the form of discretised target profile 310 (coil current reference for the TMS system), initial capacitor voltages of the switching modules 10, and all the circuit parameters (e.g., various inductance, capacitance, resistance values, initial voltages and currents, blanking interval, etc.) and MPC parameters (e.g., length of window, cost weightages, etc.), using which all A_{n,T} matrices are calculated and stored, in step 1210.

Next, in steps 1215 and 1220, the two rate limiting passes are applied on the target profile 310 to get the reference r which is then padded with Nₕ zeroes at the end.

In step 1230, the initial branch is assumed to be the zero-output-voltage branch b_{z} corresponding to u=0 and the initial accumulated cost c* is assumed infinity. With the circuit states and switching counter initialised to zero, the algorithm enters an iterative loop where, at each time-step, the mpc( ) function finds the least cost path along the MPC window in step 1235, and the step( ) function advances the circuit states by one time-step using the first branch of the least cost path in step 1240.

Thus, for respective time-steps of the control sequence 50 subsequent to the first, the step 1250 of selecting paths successively comprises initially selecting paths that commence with the overlapping part of the optimal path p* that was determined for a previous time-step of the control sequence 50 to produce an initial cost c*. After the loop is evaluated for each discrete time-step (sample) of the target profile 310, step 1245 terminates the algorithm and step 1260 returns a sequence of switching states comprising the least cost branch at each time-step.

1. A method of deriving an optimised control sequence for a pulse system comprising a reactive circuit including a pulse coil for generating electromagnetic pulses, and a switching circuit comprising plural switches arranged to switchably connect at least one capacitive discharge element to provide an output voltage that is supplied as an input to the reactive circuit, and the control sequence is a sequence of switching states of the switches over time-steps, wherein the method is performed offline prior to operating the pulse system under the control of the derived, optimised control sequence, and uses a discrete-time model of the pulse system configured to predict an electrical state of the pulse system,
the method comprising:
receiving a target profile for an electromagnetic pulse delivered by the pulse coil; and
for respective time-steps of the control sequence taken in succession,
determining an optimal path of switching states over a window of time-steps starting with the respective time-step, which optimal path of switching states optimises a cost function having a deviation cost contribution representing deviation of an electrical state of the pulse system predicted by the discrete-time model from the target profile over the window of time-steps, and
selecting the switching state of the determined path at the respective time-step as the switching state of the control sequence at the respective time-step.

2. A method according to claim 1, wherein the step of determining an optimal path of switching states comprises
performing a search of possible paths of switching states over the window by selecting paths successively and:
for an initially selected path, accumulating a total value of the cost function over the entirety of the path; and
for subsequently selected paths, accumulating a value of the cost function over successive time-steps of the path, wherein
at each time-step before the final time-step, the accumulated value of the cost function is compared to a minimum total value of the cost function accumulated over the entirety of a path previously in the search, and
if the accumulated value has reached the minimum value then the accumulation is terminated, and otherwise the accumulation is continued, and
on completion of the search, determining that the path having a minimum total value of the cost function accumulated over the entirety of the path is the optimal path.

3. A method according to claim 2, wherein, for respective time-steps of the control sequence subsequent to the first, the step of selecting paths successively comprises initially selecting paths that commence with the overlapping part of the optimal path that was determined for a previous time-step of the control sequence.

4. A method according to claim 2 or 3, wherein, at an early stage of the search, the step of selecting paths successively comprises selecting paths corresponding to diverse values of the output voltage.

5. A method according to any one of the preceding claims, wherein the cost function further has at least one further cost contribution that represents a penalty for undesirable operational characteristics of the switching circuit.

6. A method according to claim 5, wherein the at least one further cost contribution includes a voltage cost contribution that is dependent on the voltage of the at least one capacitive discharge element.

7. A method according to claim 6, wherein the voltage cost contribution represents a penalty for variation between the voltages of capacitive discharge elements.

8. A method according to claim 6 or 7, wherein the voltage cost contribution takes a prohibitive value when the voltage of a capacitive discharge element exceeds an overcharge threshold.

9. A method according to any one of the preceding claims, wherein the at least one further cost contribution includes at least one switching state cost contribution that is dependent on the switching states of the switches.

10. A method according to claim 9, wherein the at least one switching state cost contribution includes at least one switching action cost contribution representing penalties for undesirable switching actions.

11. A method according to claim 10, wherein the at least one switching action cost contribution comprises one of more of:
a switching action cost contribution representing a penalty for switching states connecting the at least one capacitive discharge element to provide the output voltage;
a switching action cost contribution representing a penalty for simultaneous change of the switching state of plural switches; and/or
a switching action cost contribution representing a penalty for changes in the output voltage at each time-step.

12. A method according to any one of claims 9 to 11, wherein the at least one switching state cost contribution includes an equalisation cost contribution representing a penalty for unequal switching counts of different switches.

13. A method according to any one of the preceding claims, wherein the discrete-time model of the pulse system is a transformation of a continuous-time model of the pulse system.

14. A method according to any one of the preceding claims, wherein the discrete-time model is a discrete-time state-space model.

15. A method according to any one of the preceding claims, wherein
the control sequence is a sequence of switching states of the switches over time-steps with insertion of blanking intervals between switching of the switches, and
the discrete-time model of the pulse system models the insertion of the blanking intervals.

16. A method according to any one of the preceding claims, wherein the method comprises adjusting the target profile before determining the paths of switching states for respective time-steps in a manner that restricts magnitudes of rates of change of the target profile.

17. A method according to any one of claims 1 to 15, wherein the target profile is a target profile of current through the pulse coil, and the electrical state of the pulse system predicted by the discrete-time model includes a current through the pulse coil.

18. A method according to claim 17, wherein the method comprises adjusting the target profile before determining the paths of switching states for respective time-steps in a manner that restricts magnitudes of rates of change of current of the target profile.

19. A method according to claim 18, wherein the step of adjusting the target profile comprises applying a predetermined limit to the change in current in each time-step that is equal to the product of the length of the time-step and the quotient of the initial output voltage divided by the inductance of the reactive circuit, or a predetermined fraction of that product.

20. A method according to claim 18 or 19, wherein the step of adjusting the target profile comprises:
predicting the output voltage at each time-step from the target profile using a model of the transfer of energy stored in the at least one capacitive discharge element to the reactive circuit, and
applying a predetermined limit to the change in current in each time-step that is equal to the product of the length of the time-step and the quotient of the predicted output voltage at each time step divided by the inductance of the reactive circuit, or a predetermined fraction of that product.

21. A method according to any one of the preceding claims, wherein the electrical state of the pulse system predicted by the discrete-time model includes variable electrical parameters of the reactive circuit.

22. A method according to any one of the preceding claims, wherein the electrical state of the pulse system predicted by the discrete-time model includes a voltage of the at least one capacitive discharge element.

23. A method according to any one of the preceding claims, wherein the reactive circuit further comprises a filter circuit arranged to filter the input to the reactive circuit before application to the pulse coil.

24. A method according to any one of the preceding claims, wherein the at least one capacitive discharge element comprises plural capacitive discharge elements and the output voltage is a multi-level output voltage.

25. A method according to claim 24, wherein the switching circuit comprises a plural switching modules, each switching module comprising an comprising a capacitive discharge element and switches arranged to connect a capacitive discharge element across a module output, the switching module outputs being cascaded to provide the multi-level output voltage.

26. A method according to claim 25, wherein the switches in each switching module are arranged in a bridge arrangement.

27. A method according to claim 25 or 26, wherein the switching modules have switching states including a forward switching state connecting the capacitive discharge element across the module output in a forward direction, a reverse switching state connecting the capacitive discharge element across the module output in a reverse direction, and at least one bypass switching state not connecting the module output across the module output.

28. A method according to any one of the preceding claims, wherein the reactive circuit is a transcranial magnetic stimulation circuit and the electromagnetic pulses are transcranial magnetic stimulation pulses.

29. A method of operating a pulse system comprising a reactive circuit including a pulse coil for generating electromagnetic pulses, and a switching circuit comprising plural switches arranged to switchably connect at least one capacitive discharge element to provide an output voltage as an input to the reactive circuit, the method comprising:
deriving an optimised control sequence for the pulse system that is a sequence of switching states of the switches over time-steps by a method according to any one of the preceding claims; and
operating the pulse system under the control of the derived, optimised control sequence.

30. A computer program capable of execution by a computer apparatus and configured, on execution, to cause the computer apparatus to perform a method according to any one of claims 1 to 28.

31. A computer-readable storage medium storing a computer program according to claim 30.

32. A computer apparatus configured to perform a method according to any one of claims 1 to 28.
